(19) United States
(12) Patent Application Publication
(10) Pub. No.: US 2018/0125442 A1
(43) Pub. Date: May 10, 2018

(54) COMPUTED TOMOGRAPHY (CT)-BASED ELASTOGRAPHY

(71) Applicant: OHIO STATE INNOVATION FOUNDATION, Columbus, OH (US)

(72) Inventors: Arunark Kolipaka, Delaware, OH (US); Rick Layman, Dublin, OH (US); Richard D. White, Columbus, OH (US)

(21) Appl. No.: 15/570,326

(22) PCT Filed: Apr. 13, 2016
(86) PCT No.: PCT/US2016/027311

§ 371 Date: Oct. 28, 2017

Related U.S. Application Data
(60) Provisional application No. 62/154,497, filed on Apr. 29, 2015.

(51) Int. Cl. A61B 6/00; A61B 6/03; A61B 5/00; G06T 7/00

(52) U.S. Cl. CPC A61B 6/5217; A61B 6/032; A61B 6/4435; G06T 2207/30024; A61B 5/0051; G06T 7/0016; G06T 2207/10081; A61B 6/5205

(57) ABSTRACT

Systems and methods are provided for CT-based elastography. A CT gantry is rotated while acquiring CT image data at a first frequency while tissue vibration is induced at a second vibrational frequency. The data acquisition frequency and the vibrational frequency are harmonically related and are synchronized such that the vibrational period aligns with the data acquisition period. Displacement of each of a plurality of tissue points are calculated in each of a series of images and a displacement map is generated demonstrating relative tissue stiffness.

### RELATED APPLICATIONS

This application claims the benefit of U.S. Provisional Application No. 62/154,497, entitled “COMPUTED TOMOGRAPHY (CT)-BASED ELASTOGRAPHY, filed Apr. 29, 2015, the entire contents of which are incorporated herein by reference.

### BACKGROUND

The present invention relates to systems and methods for evaluating the elasticity of biological tissue. In particular, the invention relates to systems for performing elastography using CT imaging modalities.

### SUMMARY

Elastography is a noninvasive technique to estimate stiffness of soft tissue. Magnetic resonance elastography (MRE) and ultrasound elastography (UE) estimate tissue stiffness to diagnose different diseases such as staging liver fibrosis, cancerous tumors, etc. However, MRE has functional deficiencies in that it: (1) cannot be used to effectively evaluate hard tissues (e.g., bone); (2) is limited by low temporal resolution; and (3) requires long scan times. UE is similarly limited by its being inapplicable for evaluation of hard tissues, as well as by low spatial resolution precluding creation of spatial stiffness maps and by limited field-of-view for full-organ imaging.

The systems and methods described herein present a novel mechanism for performing elastography using computed tomography (CT) imaging modalities. CT elastography (CTE) is a displacement-based method that estimates local displacement of tissue, in response to a mechanical stimulus, from high resolution images. This “displacement map” can be used as a surrogate to estimate stiffness. Unlike both MRE and UE, CTE can be applied to hard structures in the body (such as bone, cartilage, and teeth), as well as soft tissues, in combination with high spatial and temporal resolution. Unlike MRE, CTE can also be used to scan patients that may not be compatible for the magnetic resonance environment from a safety standpoint (e.g., patients with pacemakers or metal implants).

In some embodiments, CTE is performed by applying external mechanical vibrations in an area of interest. In some situations (e.g., due to heart valve closures), internally induced vibration may be preferable. The induced vibratory waves are then tracked using a CT scanner over time. Finally, these images are analyzed using mathematical algorithms to estimate spatial and temporal displacement maps (a surrogate for stiffness estimate).

For the application of mechanical vibrations, CTE has advantages over MRE in that the mechanical driver/actuator used to induce tissue vibration can be constructed using metallic components for pneumatic drivers, hydraulic drivers and high-current piezo-electric drivers. Such drivers can induce high frequency vibrations beyond what is possible in an MRE environment, which are necessarily limited to constructions using non-metallic components. Furthermore, CTE can be used to develop high spatial resolution displacement maps, unlike UE.

In one embodiment, the invention provides an elastography system including a CT gantry, an x-ray source, an x-ray detector, a vibration-inducing actuator, and a controller. The x-ray tube and detector geometry are fixed opposing each other and rotate around the imaging subject. The vibration-inducing actuator is positionable in contact with the imaging subject during rotational movement of the gantry to cause vibration of tissue of the imaging subject. The controller is configured to rotate the gantry while acquiring CT data at first defined frequency and to induce vibration at a second defined frequency. The controller is also configured to synchronize the acquisition of the CT data with the induced vibrations. The first defined frequency and the second defined frequency are harmonically related such that the first defined frequency is an integer multiple of the second defined frequency or vice versa.

In some embodiments, the controller of the elastography system is further configured to acquire non-vibratory CT data by rotating the gantry without inducing any vibration and to subsequently acquire vibratory CT data by rotating the gantry while inducing vibration. For the latter acquisition, multiple temporal frames are collected to capture the different vibrational phases of the object. The non-vibratory CT data and the vibratory CT data are then compared to identify, on a frame-by-frame basis, a vibration-induced local displacement map in the CT data using, e.g., rigid or non-rigid registration. Alternatively, the vibratory CT data can be used without the non-vibratory CT data. In this case, the displacements maps are constructed by comparing the two consecutive frames of the vibratory CT data.

Other aspects of the invention will become apparent by consideration of the detailed description and accompanying drawings.

### BRIEF DESCRIPTION OF THE DRAWINGS

FIG. 1 is a block diagram of a CT elastography (CTE) system according to one embodiment.

FIG. 2 is a schematic diagram of a gantry-based CT imaging system for use with the CTE system of FIG. 1.

FIG. 3 is a schematic diagram of a vibration inducement system for use with the CTE system of FIG. 1.

FIG. 4 is a flow-chart of a method for performing CTE using the system of FIG. 1.

FIG. 5 is a flow-chart of a method for performing elastography analysis and for generating a displacement map using the system of FIG. 1.

FIG. 6 is a flow-chart of another method for performing CTE including comparing vibratory CT image data to another vibratory CT image using the system of FIG. 1.

FIG. 7A is a CT image of a tissue of interest.

FIG. 7B is a displacement map generated for the tissue of interest of FIG. 7A using the method of FIG. 5.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways.

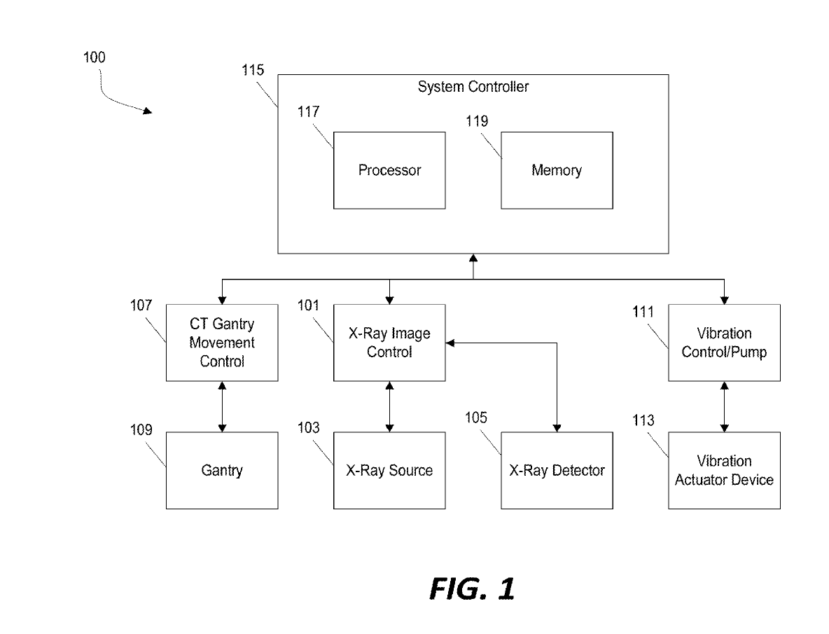

FIG. 1 illustrates an example of a system for performing elastography analysis using CT imaging. The CT elastography (CTE) system 100 includes an x-ray image control 101 that controls the operation of an x-ray source 103 and an x-ray detector 105. A CT gantry movement control system 107 controls the movement of a mechanical gantry 109. A vibration control/pump system 111 control the operation of a vibration-inducing actuator device 113. The specific operating parameters of the x-ray source 103, x-ray detector 105, gantry 109, and the vibration-inducing actuator 113 are coordinated by a system controller 115 that includes a processor 117 and a memory 119. The memory 119 is a non-transitory computer-readable memory such as, for example, hard-disk, Flash, or other RAM or ROM memory platforms. The processor 117 executes computer-readable instructions stored on the memory 119 to provide the functionality of the system 100 including, for example, the functionality as described herein.

In some implementations, the system controller 115 is only responsible for controlling the operation of the system components described above. In such implementations, CT image data acquired by the x-ray detector 105 is provided to the system controller 115 and stored to the memory 119 or an external memory such as, for example, a remote computer server or cloud computing environment. Analysis of the data are performed by a separate computer system and viewed by a medical professional. However, in other implementations, the data processing is performed by the system controller 115 using instructions stored on the memory 119.

The example of FIG. 1 illustrates the CT gantry movement control 107, the x-ray image control 101, and the vibration control 111 as components that are separate from the system controller 115. In this example, each sub-system control component 101, 107, 111 is implemented as a separate control system including its own processor, memory, and input/output (I/O) module for communicating with the system controller 115 and each respective hardware system 103, 105, 109, 113. However, in other implementations, the sub-system controllers may be either wholly or partially integrated into the system controller 115. For example, in some implementations, the system controller 115 directly provides control outputs to the motors that control movement of the gantry while, in other implementations, the system controller 115 may instead provide control instructions or parameters to a gantry movement controller, which then provides direct control instructions to the gantry motors. As such, although the discussion below addresses each sub-system controller 101, 107, 111 as a component that is separate from the system controller 115, in some implementations, the specific functionality described below may be divided differently among more or fewer controllers.

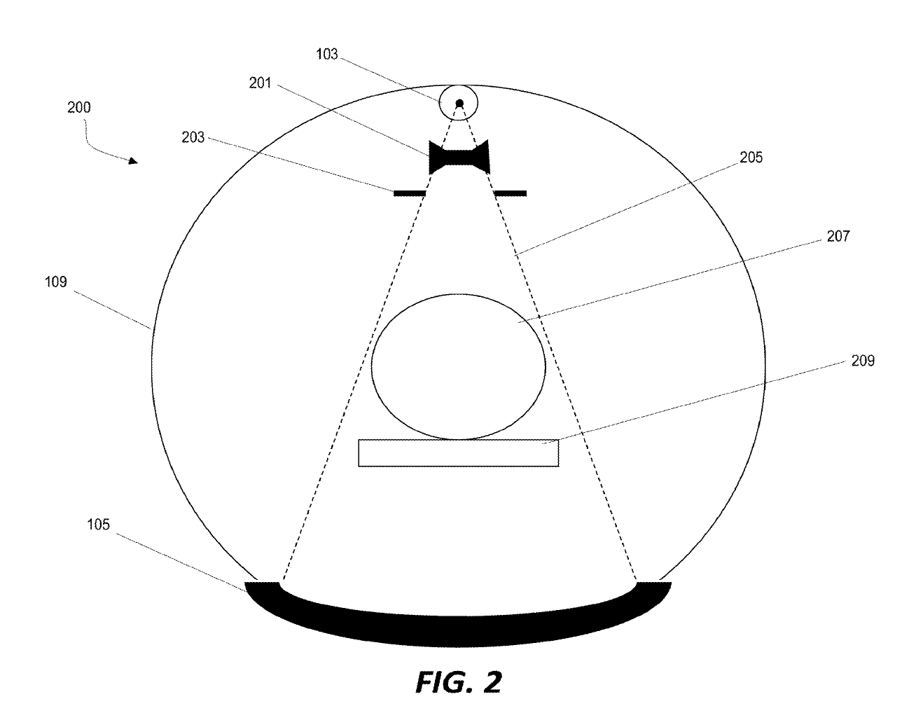

FIG. 2 illustrates an example of a gantry-based CT imaging system 200 for use as part of the system of FIG. 1. The CT system 200 includes an x-ray tube 103 which generates and emits x-rays (i.e., an x-ray source) that is coupled to one side of a gantry 109. The emitted x-rays pass through a bow-tie filter 201 and a collimator 203 to form an x-ray cone beam 205, which passes through an imaging subject 207 positioned on a CT table/platform 209 before ultimately reaching an x-ray detector 105 positioned on the opposite side of the gantry 109. The gantry 109 is controllably rotated and images are captured by the x-ray detector at a designated/controlled frequency. Because the x-ray source 103 and the x-ray detector 105 are fixedly coupled to the gantry, they rotate around the imaging subject 207 to collect x-ray image data from various perspectives around the patient while remaining positioned opposite each other. In some CT systems 200, the gantry 109 may also be moved linearly to collect data corresponding to “slices,” which are then assembled by a CT image processing system to generate a three-dimensional CT model of the imaging subject.

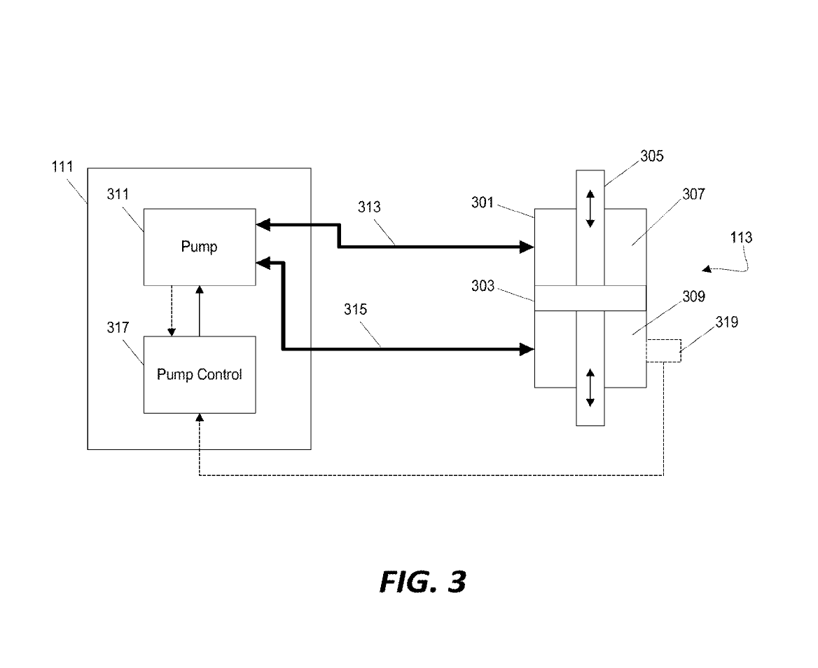

Elastography is an imaging technique that maps the elastic properties of various tissues of an imaging subject. Vibration is induced in the imaging subject while image data are captured. The captured image data are then processed to detect changes in the tissue due to the induced vibration. FIG. 3 illustrates an example of one type of vibration-inducing actuator system for inducing vibration for elastography analysis. A vibration-inducing actuator device 113 includes a cylinder housing 301 and a piston plate 303 fixedly coupled to a control rod 305. The piston plate 303 separates the internal volume of the cylinder 301 into an upper portion 307 and a lower portion 309.

The vibration-inducing actuator device 113 is coupled to a vibration control system 111 that includes a pump 311 and a pump controller 317. The pump 311 can include, for example, a hydraulic pump or a pneumatic pump. In a hydraulic pump-based system, the pump 311 provides fluid to the upper portion 307 of the cylinder 301 through a first hydraulic line 313 while at the same time drawing fluid from the lower portion 309 of the cylinder 301 through a second hydraulic line 315. This causes the piston plate 303 to move linearly in a first direction (i.e., downward in the example of FIG. 3). Conversely, when the pump 311 provides fluid to the lower portion 309 of the cylinder 301 while drawing fluid from the upper portion 307 of the cylinder 301, the piston plate 303 moves linearly in a second direction opposite the first direction (i.e., upward in the example of FIG. 3). By alternatingly pumping and drawing fluid from either side of the piston plate 303, the pump control system 111 causes alternating linear movement of the piston plate 303 and vibration of the actuator 113.

To induce vibration in tissue of a subject, the vibration-inducing actuator 113 is placed in contact with the skin of an imaging subject 207 laying on the CT table 209 (see, FIG. 2). Vibration of the actuator 113 causes similar vibration of nearby tissue in the imaging subject 207. By alternating movement of the piston plate 303 at a defined frequency, the pump control system 111 is able to induce vibration of the imaging subject tissue at the same defined frequency while image data are captured by the x-ray detector 105.

Further details of examples of vibration-inducing actuators for elastography are described in U.S. Publication No. 2013/0303882, entitled “Hydraulically-Powered System and Method for Achieving Magnetic Resonance Elastography,” and International Patent Application No. PCT/US2015/060717, filed Nov. 13, 2015 and entitled “Hydraulically-Powered and Hybrid Hydraulic-Pneumatic Systems and Methods for Achieving Magnetic Resonance Elastography,” the entire contents of both of which are incorporated herein by reference.

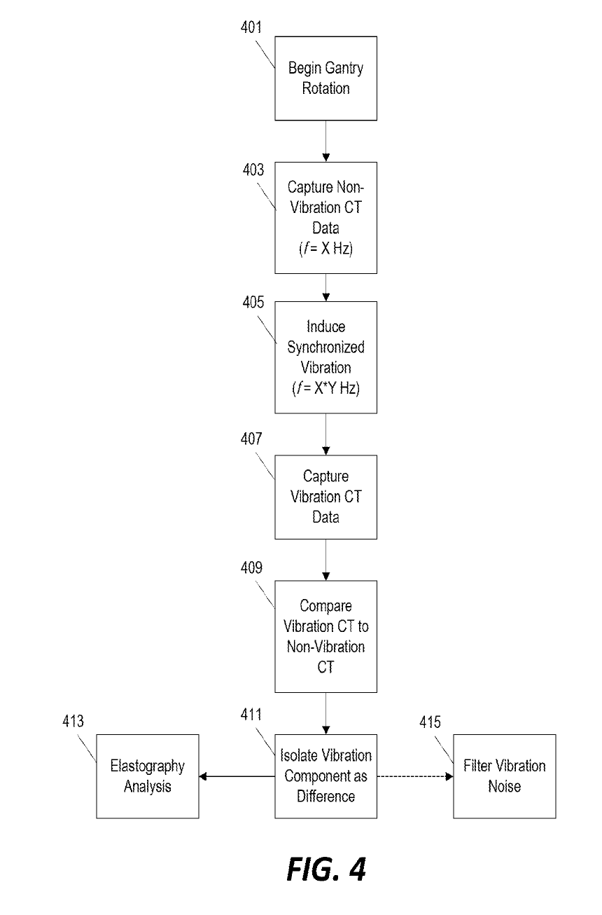

FIG. 4 illustrates an example of a method for performing CT-based elastography using the system of FIGS. 1-3. As noted above, the memory 119 of the system controller 115 stores instructions that are executed by the processor 117 and communicates with any connected sub-system controllers (e.g., 101, 107, 111) to provide the functionality described below.

When an imaging subject 207 is properly positioned on the CT table 209, the system begins rotating the imaging gantry 109 (step 401). The CT system begins capturing non-vibratory CT data from the x-ray detector 105 at a first frequency f_{d} (step 403). In some constructions, the frequency of CT data acquisition is defined by a first waveform generator or based on the hardware limits of the gantry rotation. The peak signal or some percentage of the peak signal is used to trigger the data acquisition at multiple time points (e.g., data can be captured after the prescribed delay of detected peak signal).

After sufficient non-vibratory CT data are acquired, the system activates the vibration-inducing system and induces vibration into the subject tissue at a second defined frequency fᵥ (step 405). The system then captures vibratory CT image data while vibration is being induced (step 407). In some implementations, the vibration frequency fᵥ is defined based on the first frequency f_{d} such that fᵥ is an integer multiple of f_{d}(e.g., f_{d}=X Hz; fᵥ=X*Y Hz where Y is an integer). For example, if CT data are collected at a frequency of 2 Hz, vibration of the actuator may be set at 2 Hz (Y=1), 4 Hz (Y=2), 6 Hz (Y=3), and so forth. Alternatively, the vibration frequency fᵥ and the first frequency f_{d} may be defined such that f_{d} is an integer multiple of fᵥ. The specific frequency multiple of vibration frequency fᵥ may be defined based on the type of tissue being examined or may be varied to evaluate how the elastic response of the imaging subject's tissue changes at various vibration frequencies. The vibration of the actuator is carefully controlled to synchronize the vibration frequency f with the data collection frequency f_{d} and, in some constructions, external triggering (such as, for example, cardiac triggering) is used to regulate both data collection and induced vibration. As a result, multiple offsets of the vibrations in the tissue are captured. Because the vibration frequency is defined as a multiple of the rotation frequency (or vice versa), the period of each vibration and each rotation are automatically aligned, allowing segmented acquisition, which spans multiple gantry rotations.

After sufficient vibratory and non-vibratory CT data are captured and pre-processing is performed (e.g., reconstruction of three-dimensional CT models from the captured CT data). The vibratory and non-vibratory CT data are compared to each other to identify and evaluate the elastic response of the tissue of the imaging subject (step 409). In some cases, the comparison of the vibratory and non-vibratory CT data is used to isolate the vibrational component of the CT image data (step 411). After the vibrational component is identified, elastographic analysis can be performed either automatically by the system or with the aid and intervention of a medical professional (step 413). Furthermore, in some implementations, image processing techniques are performed to filter out vibrational noise from the vibratory CT data (step 415).

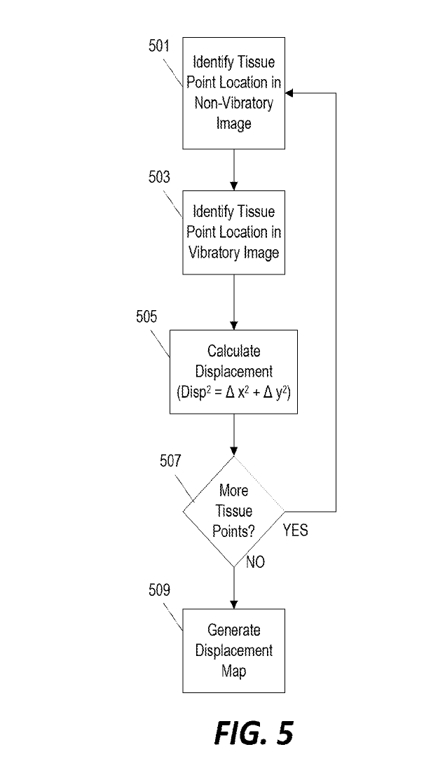

In some constructions, the “elastographic analysis” (step 413 of FIG. 4) includes using the CT data collected during the method of FIG. 4 to generate a “displacement map” that is indicative of vibrations observed in various parts of the subject tissue. FIG. 5 illustrates one such example. In particular, the method of FIG. 5 uses the collected and synchronized CT data to construct a “displacement map” of a subject tissue. The displacement map is indicative of how much a particular portion of the tissue moved under induced vibration. After the vibratory and non-vibratory CT data are collected, a specific tissue point is identified in the non-vibratory CT data (step 501). A location of the same tissue point is then identified in the vibratory CT data (step 503). The location of the tissue point can be identified and tracked in the various different CT images by monitoring a feature, such as, for example, a boundary in the CT image, traced across the multiple frames of vibratory CT data to generate a measure of local displacement.

The location of the tissue point is identified in both the non-vibratory CT image and in each vibratory CT image using two- or three-dimensional Cartesian coordinates (i.e., an X, Y and Z value). A displacement for the tissue point is then calculated for each vibratory CT image using the equations:

2D-Displacement²=Δx²+Δy²

3D-Displacement²=Δx²+Δy²+Δz²

where Δx is the difference between the x-coordinate of the tissue point location in the non-vibratory CT image (or of a reference point in the vibratory CT data) and in one of the vibratory CT images, Δy is the difference between the y-coordinate of the tissue point location in the non-vibratory CT image (or of a reference point in the vibratory CT data) and in one of the vibratory CT images, Δz is the difference between the z-coordinate of the tissue point location in the non-vibratory CT image (or of a reference point in the vibratory CT data) and in one of the vibratory CT images.

The displacement is calculated for each of a plurality of tissue points in the image data (step 507). In some embodiments, displacement is calculated for each pixel in the non-vibratory CT image. In other embodiments, an analysis resolution is defined such that only a sampling of tissue points is identified and analyzed. Once a displacement is calculated for each tissue point, a two- or three-dimensional displacement map is generated based on the non-vibratory CT image (step 509) where the color or amplitude of each point on the CT image is defined by the calculated displacement of the corresponding tissue point, which is used as a surrogate for stiffness measurement.

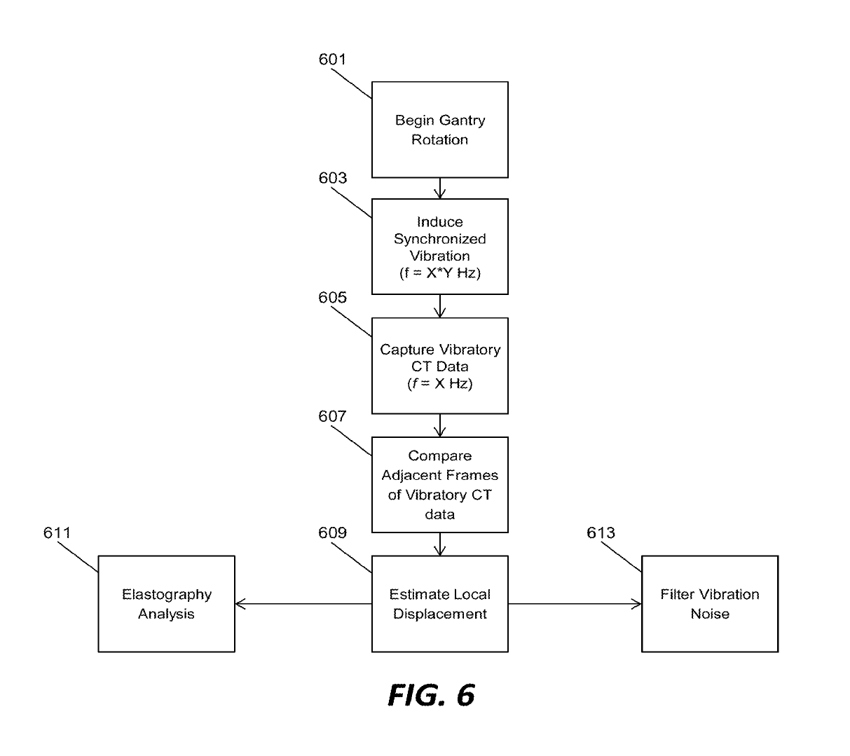

FIG. 6 illustrates an alternative method. In FIGS. 4-5, the displacement is calculated by comparing vibratory CT data to non-vibratory CT data. In contrast, in the method of FIG. 6, displacement is calculated by comparing a vibratory CT data image to another vibratory CT data image captured at a different time. After gantry rotation is initiated (step 601), the system begins to induce synchronized vibration (step 603) and captures vibratory CT data (step 605). The system in this example compares adjacent frames of vibratory CT data (step 607) and estimates local displacement based on differences between data points in the adjacent CT data frames (step 609). The system then performs elastography analysis (step 611), which, as discussed above, may include generating a displacement map. In some embodiments, the system also applies various filtering techniques (step 613) to the CT image data to remove system noise.

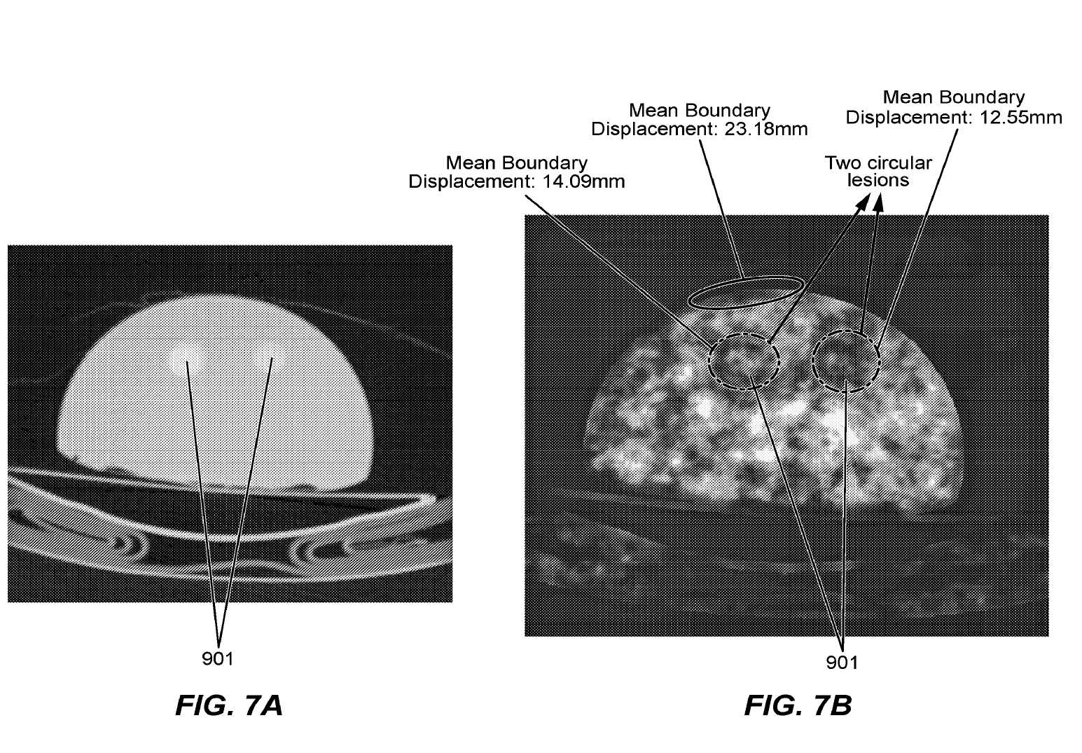

FIGS. 7A and 7B illustrate a specific example of how CTE systems and methods, such as those described above, can provide improved information regarding stiffness of a target tissue. FIG. 7A illustrates the non-vibratory CT image of a larger area of the gel phantom representing including tissue area with two circular lesions 901. However, using this CT data alone, it is difficult to detect or confirm the mechanical properties of the two circular lesions 901. FIG. 7B illustrates a generated displacement map of the same tissue where the color of each pixel is defined based on the maximum displacement for the corresponding tissue point as determined by the method of FIG. 5. The displacement map of FIG. 7B more clearly shows that two circular areas, which belong to stiffer inclusions, exhibit notably less maximum displacement than the surrounding tissue.

The specific techniques and methods discussed above are only some examples and other implementations are possible. For example, in some other embodiments the displacement map may be generated based on other techniques including estimating maximum displacement just from the raw CT data.

Furthermore, as noted above, some tissues (and some specific types of tissue irregularities) are better detected at different vibrational frequencies. Although the examples above discuss calculating displacement at only a single vibrational frequency, in some other implementations, the system may be configured to automatically and/or controllably vary the frequency of both data acquisition and induced vibration. By doing so, the system can identify a frequency where the displacement of different tissue points exhibits greater variation (i.e., a greater standard deviation) and generate a displacement map corresponding to a frequency that better differentiates the displacement of the tissue points.

Additionally, the examples above discuss synchronizing the frequency of the induced vibrations with a data acquisition frequency. In some implementations, the vibrational frequency and the data acquisition frequency can be synchronized without any adjustment to the speed or rotational frequency of the CT system gantry. However, in other implementations where the speed and rotational frequency of the gantry can be controllably regulated by the controller, the frequency of the induced vibration and the rotation of the gantry may be synchronized in order to provide the vibrational CT data as discussed above.

Thus, the invention provides, among other things, a system and method for performing elastographic imaging and analysis using CT imaging data by synchronizing the data acquisition frequency of a CT system with a vibrational frequency of a vibration-inducing actuator device. Various features and advantages of the invention are set forth in the following claims.

What is claimed is:

1. An elastography system comprising:
a CT gantry positionable to rotate around an imaging subject;
an x-ray source fixedly coupled to the CT gantry on a first side of the imaging subject;
an x-ray detector fixedly coupled to the CT gantry on a second side of the imaging subject, the second side being opposite the first side;
a vibration-inducing actuator positionable in contact with the imaging subject during rotational movement of the gantry; and
a controller configured to
acquire CT data at a first defined frequency as the gantry rotates around the imaging subject;
cause vibration of the vibration-inducing actuator at a second defined frequency, wherein one of the first defined frequency and the second defined frequency is defined as an integer multiple of the other; and
synchronize acquisition of the CT data with vibration of the vibration-inducing actuator while acquiring CT data from the x-ray detector.

2. The elastography system of claim 1, wherein the controller is further configured to calculate a displacement for each of a plurality of tissue points in the acquired CT data, wherein the displacement is indicative of a change in position of a tissue point from a first CT data image compared to a second CT data image.

3. The elastography system of claim 2, wherein the controller is further configured to generate a displacement map, wherein a color of each pixel in the displacement map is defined based on the calculated displacement for a corresponding tissue point.

4. The elastography system of claim 2, wherein the controller is further configured to calculate the displacement of each of the plurality of tissue points using the equation
Displacement²=Δx²+Δy²
where Δx is a difference between an x-axis location of the tissue point in the first CT data image and an x-axis location of the tissue point in the second CT data image, and where Δy is a difference between a y-axis location of the tissue point in the first CT data image and a y-axis location of the tissue point in the second CT data image.

5. The elastography system of claim 2, wherein the controller is further configured to calculate the displacement of each of the plurality of tissue points using the equation
Displacement²=Δx²+Δy²+Δz²
where Δx is a difference between an x-axis location of the tissue point in the first CT data image and an x-axis location of the tissue point in the second CT data image, where Δy is a difference between a y-axis location of the tissue point in the first CT data image and a y-axis location of the tissue point in the second CT data image, and where Δz is a difference between a z-axis location of the tissue point in the first CT data image and a z-axis location of the tissue point in the second CT data image.

6. The elastography system of claim 2, wherein the first CT data image is a CT data image acquired at a first time while vibration is induced in the tissue and the second CT data image is a CT data image acquired at a second time, subsequent to the first time, while vibration is induced.

7. The elastography system of claim 2, wherein the first CT data image is a CT data image acquired while vibration is not induced in the tissue and the second CT data image is a CT data image acquired while vibration is induced in the tissue.

8. The elastography system of claim 1, wherein the controller is further configured to
acquire non-vibratory CT data from the x-ray detector at the first defined frequency while rotating the gantry and not causing any vibration of the vibration-inducing actuator;
acquire vibratory CT data from the x-ray detector at the first defined frequency while rotating the gantry and causing vibration of the vibration-inducing actuator at the second defined frequency; and
compare the non-vibratory CT data and the vibratory CT data to identify a vibratory component of the CT data.

9. The elastography system of claim 1, wherein the controller is configured to synchronize acquisition of the CT data with vibration of the vibration-inducing actuator by controlling the vibration-inducing actuator such that a vibratory period of the induced vibration aligns with a CT data acquisition period.

10. The elastography system of claim 9, wherein the first defined frequency is defined and the CT data acquisition is synchronized using an external trigger.

11. The elastography system of claim 1, wherein the vibration-inducing actuator includes one selected from a group consisting of a pneumatic actuator, a hydraulic actuator, and a piezo-electric actuator, and wherein the vibration-inducing actuator includes metallic components.

12. A method for performing elastography analysis using CT image data, the method comprising:
acquiring CT image data at a first defined frequency using an x-ray detector coupled to a rotating CT gantry;
inducing vibration of a target tissue by controllably vibrating a vibration-inducing actuator at a second defined frequency, the first defined frequency and the second defined frequency being harmonically related, the vibration-inducing actuator being positioned in contact with an imaging subject during rotational movement of the rotating CT gantry; and
synchronizing acquisition of the CT data with vibration of the vibration-inducing actuator while acquiring CT data from the x-ray detector.

13. The method of claim 12, further comprising calculating a displacement for each of a plurality of tissue points in the acquired CT data, wherein the displacement is indicative of a change in position of a tissue point from a first CT data image compared to a second CT data image.

14. The method of claim 13, further comprising generating a displacement map, wherein each pixel in the displacement map is indicative of the calculated displacement for a corresponding tissue point.

15. The method of claim 13, wherein calculating the displacement for each of the plurality of tissue points in the acquired CT data includes calculating a displacement of each of the plurality of tissue points using the equation
Displacement²=Δx²+Δy²
where Δx is a difference between an x-axis location of the tissue point in the first CT data image and an x-axis location of the tissue point in the second CT data image, and where Δy is a difference between a y-axis location of the tissue point in the first CT data image and a y-axis location of the tissue point in the second CT data image.

16. The method of claim 13, wherein calculating the displacement for each of the plurality of tissue points in the acquired CT data includes calculating a displacement of each of the plurality of tissue points using the equation
Displacement²=Δx²+Δy²+Δz²
where Δx is a difference between an x-axis location of the tissue point in the first CT data image and an x-axis location of the tissue point in the second CT data image, where Δy is a difference between a y-axis location of the tissue point in the first CT data image and a y-axis location of the tissue point in the second CT data image, and where Δz is a difference between a z-axis location of the tissue point in the first CT data image and a z-axis location of the tissue point in the second CT data image.

17. The method of claim 13, further comprising:
generating the first CT data image using CT data acquired at a first time while vibration is induced in the tissue; and
generating the second CT data image using CT data acquired at a second time, subsequent to the first time shifted by a percentage of the second defined frequency, while vibration is induced.

18. The method of claim 13, further comprising:
generating the first CT data image using CT data acquired while vibration is not induced in the tissue; and
generating the second CT data image using CT data acquired while vibration is induced in the tissue.

19. The method of claim 12, further comprising:
acquiring non-vibratory CT data from the x-ray detector at the first defined frequency while rotating the gantry and not causing any vibration of the vibration-inducing actuator;
acquiring vibratory CT data from the x-ray detector at the first defined frequency while rotating the gantry and causing vibration of the vibration-inducing actuator at the second defined frequency; and
comparing the non-vibratory CT data and the vibratory CT data to identify a vibratory component of the CT data.

20. The method of claim 13, wherein synchronizing the acquisition of the CT data with the vibration of the vibration-inducing actuator includes controlling the vibration-inducing actuator such that a vibratory period of the induced vibration aligns with a CT data acquisition period.

21. The method of claim 20, further comprising defining and synchronizing the CT data acquisition using an external trigger.

22. The method of claim 12, wherein causing vibration of the vibration-inducing actuator at the second defined frequency includes causing vibration of a vibration-inducing actuator selected from a group consisting of a pneumatic actuator, a hydraulic actuator, and a piezo-electric actuator, and wherein the vibration inducing actuator includes metallic components.
